(19) United States
(12) Patent Application Publication
(10) Pub. No.: US 2021/0121671 A1
(43) Pub. Date: Apr. 29, 2021

(54) SOFTGEL CAPSULE FOR CONTROLLED DELIVERY OF TOPICAL AND ORAL PRODUCTS

(71) Applicants: Neil Hicks, Goldens Bridge, NY (US); Paul Fiumara, Mount Kisco, NY (US)

(72) Inventors: Neil Hicks, Goldens Bridge, NY (US); Paul Fiumara, Mount Kisco, NY (US)

(21) Appl. No.: 17/079,452

(22) Filed: Oct. 24, 2020

Related U.S. Application Data
(60) Provisional application No. 62/925,322, filed on Oct. 24, 2019.

(51) Int. Cl. A61M 35/00; A61M 31/00

(52) U.S. Cl. CPC A61M 35/003; A61M 2209/06; A61M 31/002

(57) ABSTRACT

A softgel capsule for controlled delivery of topical and oral products comprising, a substantially conical-shaped base and a top planar mouth end covered with a soft substrate with perforations to eject a measured quantity of a topical cream, lotion, gel, or other medicament on to a skin surface in a controlled manner without spattering, providing for a clean and mess-free application. The softgel capsule is also used to deliver a measured quantity of an oral medicament into the oral cavity in a controlled manner and the capsule itself is capable of being ingested after the dispersion of its contents into the oral cavity. The small size of the softgel capsule makes it easy to pack and transport when traveling.

### CROSS REFERENCE TO RELATED APPLICATION

This application claims priority to U.S. Provisional Patent Application No. 62/925,322 filed, Oct. 24, 2019 which is incorporated herein, in its entirety.

### FIELD OF THE INVENTION

The present invention is generally related to the delivery of pharmaceutical products. More particularly, the invention is related to a softgel capsule for controlled delivery of topical and oral products.

### BACKGROUND OF THE INVENTION

The use of lotions and creams to nourish and enhance the appearance of skin on the face and body is a daily routine followed by a majority of the population. Medicaments in the form of creams, gels and balms are also used to treat damaged skin. A vast number of these topical applications are packaged in bottles and tubes, and once they are opened, or their seals broken, the contents are exposed to the atmosphere, resulting in their slow and sometimes rapid decomposition due to chemical changes in their ingredients brought about by exposure to air and moisture. In addition, a significant quantity of these products are wasted due to over-application since the dosage to be used is not clearly specified on the labels affixed to the bottles and tubes. There is also the issue of product waste because of the inability to reach and/or completely empty the contents of the bottles, or tubes. In the case of oral medications, tablets in general are difficult to administer and some individuals have difficulty swallowing them.

To overcome the inherent problems of product wastage associated with packaging lotions, creams, cosmetics and medicaments in bottles and tubes, manufacturers have in recent years resorted to selling their products in single dose packages. Blister packs are popularly used for dry products such as medicinal tablets. Liquid products such as creams, serums, cosmetics, and liquid medicaments are packaged in ampules and disparate capsules by some manufacturers. Capsules holding these products come in various shapes and sizes. Some require puncturing the capsule with a sharp instrument to release the contents for application, resulting in the contents splattering and spewing out in different directions leading to a messy application. The present invention of a softgel capsule overcomes these deficiencies in the prior art of capsules.

### SUMMARY OF THE INVENTION

The present invention is a softgel capsule that is configured to hold a single dose of one of either a cream, lotion, cosmetics such as foundation, or topical medicaments such as antibiotics to treat skin lesions and infections, pain relief creams, gels and balms, and other topical applications, to rejuvenate, or treat the skin on the face, or other body parts. The softgel capsule of the invention can also be used to hold and dispense oral medications.

It is an object of the present invention to provide a softgel capsule that can be comfortably held between the thumb and forefinger to squeeze the softgel capsule and release the contents of the capsule on to a skin surface for topical applications of creams, lotions, and medicaments, or into the oral cavity for administering oral medications.

It is yet another object of the present invention to provide a softgel capsule that can be comfortably held between the thumb and forefinger through its substantially conical shaped base to squeeze the contents out through the distal substantially planar top mouth end in a controlled manner without the contents spattering, to deliver the contents of the capsule to their target whether that target is a skin surface, or oral cavity, in the case of oral medications.

Another object of the present invention of a softgel capsule is to provide a convenient means to transport a measured quantity of topical and oral medications, creams, lotions, bath gels, cosmetics and other essential liquids especially when traveling.

In the exemplary embodiment of the softgel capsule of the invention, the capsule is substantially conical-shaped with a round base and a distal top mouth end that is substantially circular, flat and planar and covered with a substrate configured with a plurality of perforations to allow the contents of the capsule to be ejected out when the round base end is squeezed between the thumb and forefinger and/or thumb and middle finger depending on convenience.

The objects, features and advantages of the present invention described in this summary of the invention will become obvious and appreciated by one skilled in the art when viewed in conjunction with the accompanying drawings, detailed description of the invention, and the appended claims.

### BRIEF DESCRIPTION OF THE DRAWINGS

FIG. 1 is a perspective view of a human hand illustrating the manner in which the softgel capsule of the present invention is held between the thumb and forefinger in the palm of the hand.

FIG. 2 is a perspective view of the softgel capsule of the present invention.

FIG. 3 is a perspective view of the top mouth end of the softgel capsule of the present invention.

FIG. 4 is a perspective view of a human hand illustrating the manner in which the softgel capsule of the present invention is squeezed between the thumb and forefinger of the palm of the hand.

FIG. 5 is a perspective view of the bottom end of the softgel capsule of the present invention.

FIG. 6 is a perspective side view of the softgel capsule of the present invention.

### DETAILED DESCRIPTION OF THE INVENTION

The present invention is a softgel capsule that can hold a single dose of an oral, or topical medication, a measured quantity of a cream, lotion, cosmetic, or other liquid or gel that can be dispensed through a gentle squeeze of the capsule between the fingers of a human hand for a targeted application.

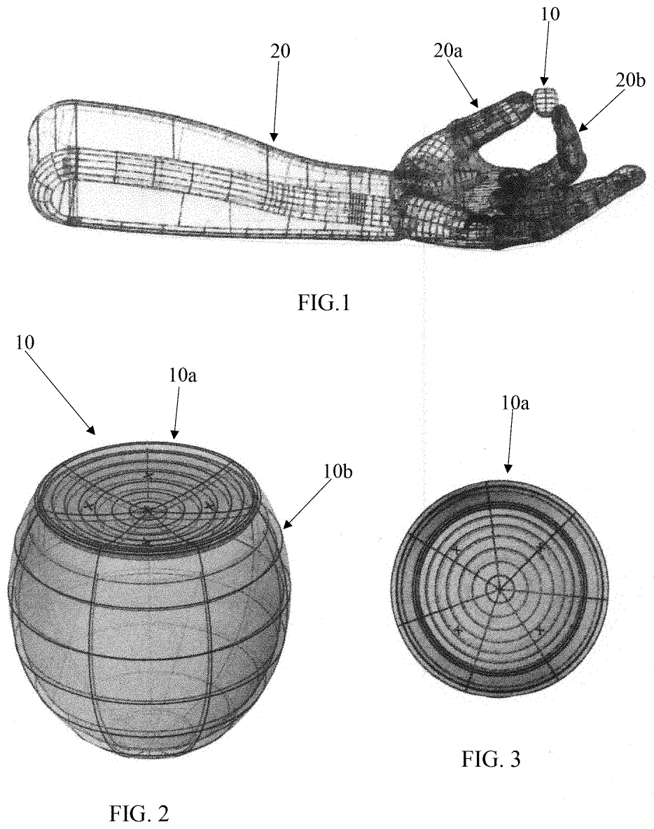

Referring now to the drawings wherein, like numerals represent like components in the several views presented and discussed, and more particularly referring now to FIG. 1 the figure, is a perspective view of a human hand 20 holding the softgel capsule 10 of the present invention. The softgel capsule 10 is sized to fit in the palm of the hand 20 between the thumb 20a and forefinger 20b of the palm of the hand 20 to facilitate squeezing of the soft gel capsule 10 to dispense the oral medication, topical medicament, cream, lotion, or other liquid and/or gel products contained within the softgel capsule 10 to the appropriate target whether it is the skin surface, or oral cavity.

FIG. 2 is a perspective view of the exemplary embodiment of the softgel capsule 10 of the present invention illustrating the substantially circular and flat, planar top mouth end 10a and the substantially conical shaped base 10b. In this embodiment of the softgel capsule 10 the circular and flat, planar top mouth end 10a is covered with a soft substrate and configured with a plurality of perforations to facilitate the quick and efficient ejection of the contents of the softgel capsule 10 to the intended target, in a controlled manner, without spattering, whether that target is a skin surface, or oral cavity in the case of oral medications.

FIG. 3 is a perspective view of the top mouth end 10a of the softgel capsule of the present invention. The top mouth end 10a is composed of a soft substrate with perforations that open when pressure is applied to the base of the softgel capsule through the act of squeezing the base of the capsule using the thumb and forefinger.

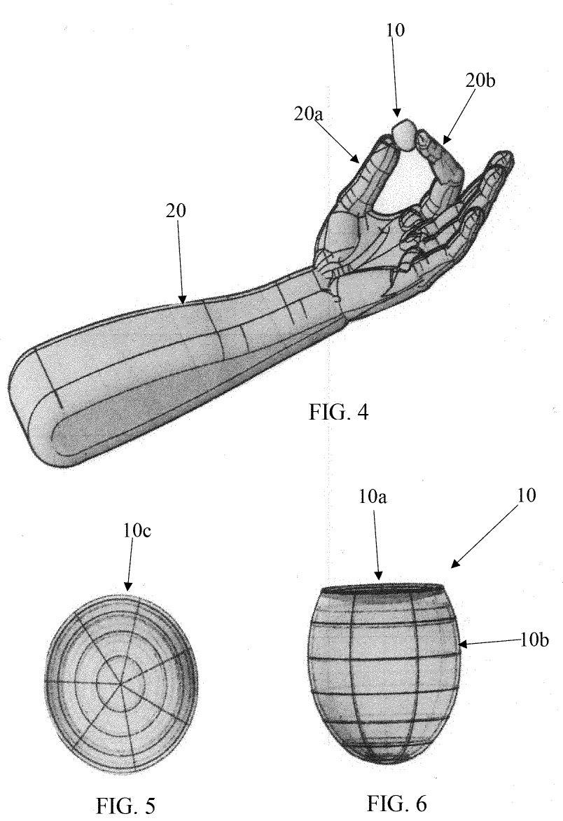

FIG. 4 is a perspective view of a human hand 20 illustrating the manner in which the softgel capsule 10 of the present invention is squeezed between the thumb 20a and forefinger 20b of the palm of the hand. After placing the softgel capsule 10 between the thumb 20a and forefinger 20b the hand 20 is brought close to the target on which the contents of the softgel capsule 10 is to be dispensed and pressure applied to the base of the softgel capsule 10 by squeezing the softgel capsule 10 base between the thumb 20a and forefinger 20b to open up the perforations on the top mouth end of the softgel capsule to release the contents of the softgel capsule 10 to the target surface. Embodiments of the softgel capsule for dispersion of oral medicaments, may also be ingested after its contents are ejected into the oral cavity.

FIG. 5 is a perspective view of the bottom end 10c of the softgel capsule of the present invention. The bottom end 10c of the base of the softgel capsule is substantially curved and oval-shaped.

FIG. 6 is a perspective side view of the softgel capsule 10 of the present invention. The softgel capsule 10 of the invention has a substantially conical-shaped base 10a with a rounded bottom end and a circular and flat, planar top mouth end 10b.

While the present invention has thus been described through its preferred embodiment, it is to be understood that the embodiment of the present invention as described herein do not limit any application or scope of the invention and that the invention can be carried out and practiced in various ways and implemented in embodiments other than the one outlined and described above. It should be further understood and obvious to one skilled in the art that alternatives, modifications, and variations of the embodiment of the present invention may be construed as being within the spirit and scope of the appended claims.

What is claimed is:

1. A softgel capsule for controlled delivery of topical and oral products, said softgel capsule comprising:
a substantially conical-shaped base;
a substantially circular and flat, planar top mouth end;
said substantially conical-shaped base having a substantially oval-shaped bottom end;
said substantially circular and flat, planar top mouth end covered with a soft substrate configured with perforations;
said softgel capsule filled with a measured quantity of one of a topical cream, liquid, gel, skin medicament, oral medicament; and
said softgel capsule sized to fit within a palm of a human hand between a thumb and a forefinger of said palm of said human hand.

2. The softgel capsule of claim 1 wherein when the softgel capsule is placed in the palm of the human hand and the substantially conical-shaped base of the softgel capsule is squeezed between the thumb and forefinger, contents of the softgel capsule are ejected through the perforations on the substrate covering the substantially circular and flat planar top mouth end of the softgel capsule.

3. The softgel capsule of claim 2 wherein when the softgel capsule is placed in the palm of the human hand and the substantially conical-shaped base of the softgel capsule is squeezed between the thumb and forefinger, contents of the softgel capsule are ejected through the perforations on the substrate covering the circular, flat planar top mouth end of the softgel capsule to a target of application as a skin surface or oral cavity in a controlled manner, without spattering, facilitating a clean and mess-free application.

4. The softgel capsule of claim 1, wherein the softgel capsule containing an oral medicament can be ingested after its contents are ejected into an oral cavity.

5. The softgel capsule of claim 1 wherein, the softgel capsule sized to fit within the palm of the human hand makes it convenient to pack and transport when traveling.
